(19) United States
(12) Patent Application Publication
(10) Pub. No.: US 2006/0259066 A1
(43) Pub. Date: Nov. 16, 2006

(54) Bifurcated artery filter system

(76) Inventor: Charles L. Euteneuer, St. Michael, MN (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 11/117,018

(22) Filed: Apr. 28, 2005

(51) Int. Cl. A61M 29/00

(52) U.S. Cl. 606/200

(57) ABSTRACT

An embolic protection system including a first occlusive device having an elongate shaft having a proximal end and distal end. A lumen extending in part through an elongate shaft. Occlusive member disposed proximate distal end of the elongate shaft. Shaft having transversely disposed opening. Second occlusive device including elongate shaft having a proximal end and a distal end. A lumen extending at least in part through the elongate shaft. An occlusive member disposed proximate the distal end of the elongate shaft of the second occlusive device. The second occlusive member being disposed proximate the distal end of the elongate shaft of the second occlusive device. The second occlusive member being disposed in part within the lumen of the first occlusive member.

### FIELD OF THE INVENTION

The present invention relates generally to the field of intravascular devices. More specifically, the present invention pertains to multiple embolic protection devices.

### BACKGROUND OF THE INVENTION

Intravascular devices such as an embolic protection filters are typically placed in a vessel such as an artery or vein to filter emboli contained in the blood stream. Examples of procedures employing such filters include angioplasty, atherectomy, thrombectomy, and stenting. These procedures generally involve transluminally inserting and delivering within the artery or vein an elongated wire and filter to a location distal a lesion. Once placed, a therapeutic device such as an angioplasty catheter is advanced along the wire to the site of the lesion to perform a therapeutic procedure (e.g. percutaneous transluminal coronary angioplasty). A stent can also be advanced to the site of the lesion and engaged along the wall of the vessel to prevent restenosis from occurring within the vessel. Although a wire is normally used to advance these devices, that need not be the only way to do so.

### SUMMARY OF THE INVENTION

The present application pertains to embolic protection devices. For example, in the situation where a lesion or target site is disposed proximally of a bifurcated vessel multiple embolic protection devices may be used to prevent or limit the flow of embolic material downstream of the bifurcation.

For example, an embolic protection system to accomplish this may include a first occlusive device having an elongate shaft having a proximal end and a distal end. A lumen can extend at least in part through the elongate shaft. An occlusive member, such as a filter, can be disposed proximal the distal end of the elongate shaft. The shaft can have a generally transversely disposed opening. A second occlusive device including an elongate shaft having a proximal end and a distal end, and including an occlusive member disposed proximate to the distal end, can be disposed at least in part within the lumen of the first occlusive member. Both occlusive devices can be advanced distally of the target site. Then one of the occlusive devices can be disposed in the main vessel and the other disposed in the branch vessel.

### BRIEF DESCRIPTION OF THE DRAWINGS

FIG. 1 is a view of an embolic protection system disposed within the aorta and renal artery;

FIG. 2 is a view of a dilatation balloon advanced over the system of FIG. 1;

FIG. 3 is a cross-sectional view of first and second occlusive devices; and

FIG. 4 is an alternative embodiment of the first and second occlusive devices of FIG. 3.

### DETAILED DESCRIPTION OF THE INVENTION

The following description should be read with reference to the drawings, in which like elements in different drawings are numbered in like fashion. The drawings, which are not necessarily to scale, depict selected embodiments and are not intended to limit the scope of the invention. Although examples of construction, dimensions, and materials are illustrated for the various elements, those skilled in the art will recognize that many of the examples provided have suitable alternatives that may be utilized.

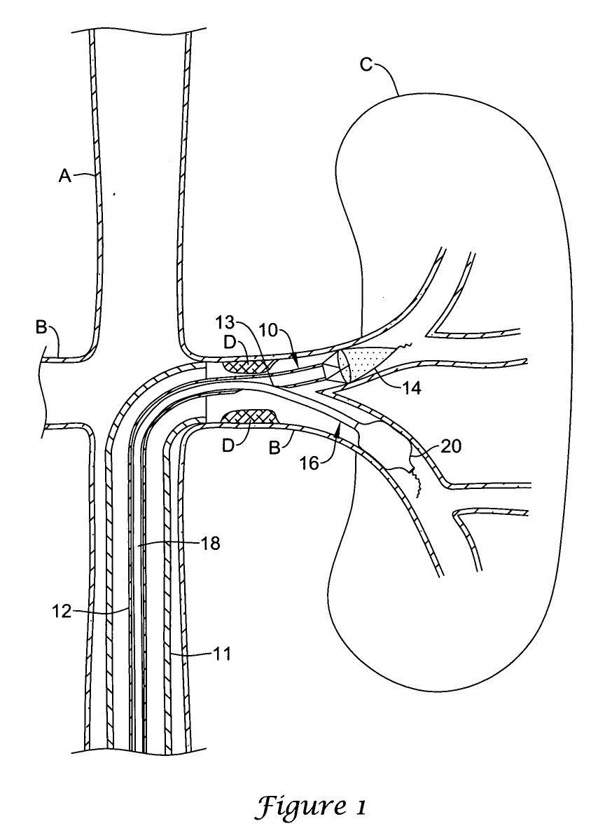

FIG. 1 is a view of aorta A, renal arteries B and kidney C. A lesion (target site) D is disposed within one of the renal arteries B. An embolic protection system including a first occlusive device 10 and a second occlusive device 16 is advanced distally of lesion D through guide catheter 11. The first occlusive device 10 includes a transverse side opening 13 through which second occlusive device 16 extends.

First occlusive device 10 includes elongate shaft 12 having a proximal end (not shown) and a distal end. A first occlusive member 14, such as a filter, is disposed proximate the distal end of elongate shaft 12. Elongated shaft 12 can be a hypotube or other tube having good pushability and steerability characteristics. Shaft 12 can have, for example, an outer diameter of between 0.015 to 0.020 inches, or for example, between 0.016 to 0.019 inches. For example, the outer diameter of shaft 12 can be about 0.018 inches.

The second occlusive device 16 includes elongate shaft 18 and having a proximal end (not shown) and a distal end. A second occlusive member 20, such as a balloon, is disposed proximate the distal end of the elongate shaft 18, and is in fluid communication with a lumen therethrough. Elongate member 18 can be a hypotube or it can be made from other materials having good pushability and steerability characteristics. The outside diameter or elongate member 18 can be for example between 0.010 to 0.015 inches, or for example, approximately 0.012 inches. The outer diameter of elongate shaft 18 is correlated with the inner diameter of shaft 12 to allow sideable movement of shaft 18 within shaft 12.

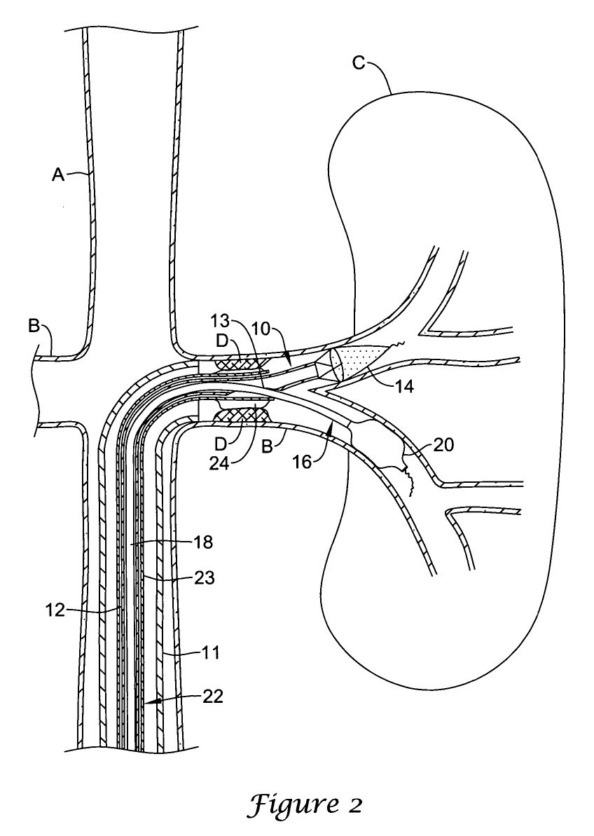

FIG. 2 is a view of the embolic protection system of FIG. 1 wherein a dilation catheter 22 has been advanced over first occlusive device 10 and second occlusive device 16. Dilation catheter 22 includes an elongate shaft 23 having a proximal end (not shown) and a distal end. A dilation balloon 24 is disposed proximate to the distal end of elongate shaft 23. Dilation balloon 22, can be, for example, of conventional construction. Dilation catheter 22 can be a single operator exchange type device, for example.

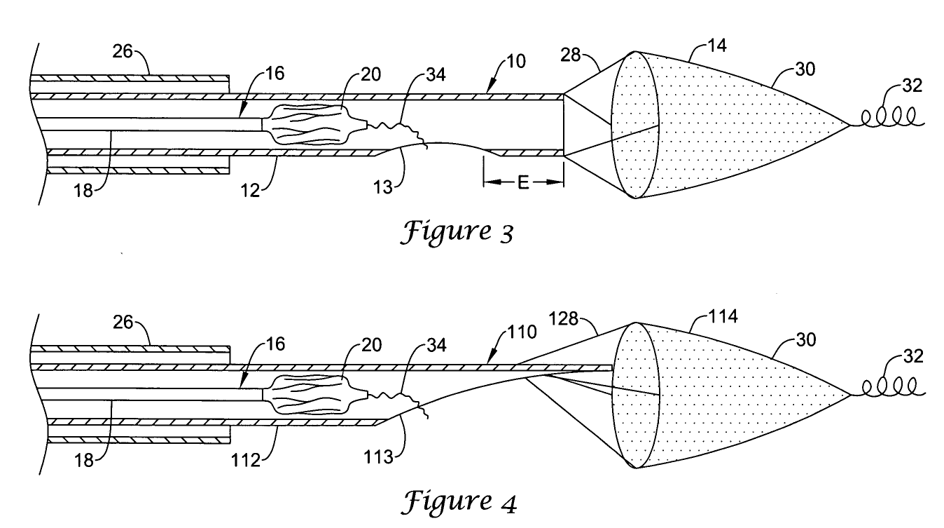

FIG. 3 is a view of a distal portion of the embolic protection system wherein second occlusive device 16 is disposed within the lumen of first occlusive device 10. A retrieval or delivery catheter 26 is disposed over first occlusive device 10. Catheter device 26 can be moved over occlusive member 14 to compress it for removal or delivery. When catheter 26 is removed from occlusive member 14 it will expand from a first compressed position to a second expanded position to transverse the vessel, for example, the renal artery. It may be desirable to use different delivery and retrieval catheters. For example, a smaller diameter delivery catheter can be used when no emboli is present within an occlusive member 14, whereas a larger diameter retrieval catheter can be used when emboli is present within occlusive member 14.

Occlusive member 14 can be, for example, an embolic protection filter. Such filters can include flexible struts 28 to create an entry opening for embolic material. Such struts 28 can be made from flexible materials such as nickel titanium alloys which can be compressed for delivery or retrieval and expanded within the vessel for filtering. A filter membrane 30 can define a plurality of exit openings sized to allow blood to pass through the membrane and retain embolic material within the filter. Various drilled sheet materials or braided fabrics, for example can be used to form filter membrane 30. It should be understood that various other materials and configurations can be used to form filter membrane 30. Occlusive member 14 can include a tip 32, such as a spring tip which can be bent to aid in steering of first occlusive device 10 through a patient's vasculature.

Transverse opening 13 can be positioned a distance E from the distal end of the elongate shaft 12. Transverse opening 13 can be located proximate the distal end of elongate shaft 12. For example, the transverse opening can be located, for example, between 0 to 5 cm from the distal end of the shaft, or for example, between 0 to 3 cm from the distal end of the elongate shaft, or for example, between 0 to 2 cm from the distal end of the elongate shaft. The transverse opening has a length and a width. The length of the transverse opening can be, for example, between 0.5 and 3.0 cm.

To enable a second occlusive device 16 to emerge from first occlusive device 10, second occlusive member 20 can include a tip 34, such as a spring tip which can be bent to steer second occlusive device 16 out through opening 13 and into branch vessel B.

FIG. 4 is an alternate embodiment of the embolic protection system where a first occlusive device 110 includes a side opening 113 at the distal end of elongate member 112. In such a configuration, the distance E from the distal end is zero. First occlusive member 114 is similar to first occlusive 14. Struts 128 are similar to struts 28 of occlusive member 114, but have been reconfigured to connect to an altered distal end of elongate shaft 112.

In use, guide catheter 11 can be advanced to the ostium of renal artery B, from a femoral artery access point. First occlusive device 10 (or 110) can be advanced through guide catheter 11 into renal artery B. Delivery sheath 26 contains first occlusive member 14 (or 114) in a compressed state until the occlusive member is positioned in the desired portion of renal artery B, distally of target site D. It should be noted that the designation of one branch or the other of renal artery B as a branch, or the renal artery itself is arbitrary. Occlusive member 14 (or 114), can be placed either in the renal artery or the branch artery.

After occlusive member 14 is positioned, delivery catheter 26 is withdrawn to allow occlusive member 14 to expand and transverse aorta B. Second occlusive member 20 can then be advanced from transverse opening 13 (or 113) into the branch artery. When second occlusive member 20 is in desirable location of the branch artery, it can be inflated to transverse the branch artery. Then dilation catheter 22 can be advanced to target site D to dilate the renal artery. It can be appreciated that other therapeutic devices could also be advanced over first occlusive 10 to treat target site D.

After the therapeutic procedure has been completed, the various devices can be withdrawn in the reserve order delivered. For example, the therapeutic catheter can be withdrawn. Then occlusive member 20 deep laded and withdrawn into occlusive device 10. Retrieval catheter can be advanced over first occlusive device 10 to compress occlusive member 14. Then first occlusive device 10 and retrieval catheter 26 can be removed. Finally, guide catheter 11 can be removed.

Having thus described the several embodiments of the present invention, those of skill in the art will readily appreciate that other embodiments may be made and used which fall within the scope of the claims attached hereto. Numerous advantages of the invention covered by this document have been set forth in the foregoing description. It will be understood that this disclosure is, in many respects, only illustrative. Changes may be made in details, particularly in matters of shape, size and arrangement of parts without exceeding the scope of the invention as described in the appended claims.

What is claimed is:

1. A method of providing embolic protection, comprising:
providing a first occlusive device including an elongate shaft having a proximal end and a distal end, a lumen extending at least in part through the elongate shaft, and an occlusive member disposed proximate the distal end of the elongate shaft;
providing a second occlusive device including an elongate shaft having a proximal end and a distal end, a lumen extending at least in part through the elongate shaft, and a occlusive member disposed proximate the distal end of the elongate shaft, and the second occlusive member being disposed at least in part within the lumen of the elongate shaft of the first occlusive member;
advancing the first occlusive member distal a target site in a renal artery; and
advancing the second occlusive device to a branch vessel of the renal artery.

2. The method of providing embolic protection in accordance with claim 1, wherein the second occlusive device is advanced to proximate the ostium of the branch vessel, while being disposed within the first occlusive device.

3. The method of providing embolic protection in accordance with claim 1, wherein the first occlusive member includes a filter.

4. The method of providing embolic protection in accordance with claim 1, wherein the occlusive member of the second occlusive device includes a balloon.

5. The method of providing embolic protection in accordance with claim 1, further comprising providing a delivery sheath disposable over the first occlusive member.

6. The method of providing embolic protection in accordance with claim 5, further comprising moving the delivery sheath proximally relative to the first occlusive member to allow the first occlusive member to expand transversely from a compressed position to an expanded position transversing the renal artery.

7. The method of providing embolic protection in accordance with claim 1, further comprising advancing the second occlusive member out of a generally transverse opening in the first occlusive device into the branch vessel.

8. The method of providing embolic protection in accordance with claim 7, wherein the second occlusive member includes a balloon.

9. The method of providing embolic protection in accordance with claim 8, further comprising of inflating the balloon to include the branch vessel.

10. An embolic protection filter, comprising:
an elongate shaft, having a proximal end and a distal end, a lumen extending at least in part therethrough;
a filter element disposed proximate the distal end of the shaft; and
the shaft having a generally transversely oriented opening into the lumen, proximate the distal end of the shaft.

11. The embolic protection filter in accordance with claim 10, wherein the shaft comprises the hypotube having an outer diameter of 0.015 to 0.020 inches.

12. The embolic protection filter in accordance with claim 11, wherein the outer diameter of the shaft is 0.016 to 0.019 inches.

13. The embolic protection filter in accordance with claim 12, wherein the outer diameter of the shaft is 0.018 inches.

14. The embolic protection filter in accordance with claim 10, wherein the transverse opening is located 0 to 5 cm from the distal end of the shaft.

15. The embolic protection filter in accordance with claim 14, wherein the transverse opening is located 0 to 3 cm from the distal end of the elongate shaft.

16. The embolic protection filter in accordance with claim 15, wherein the transverse opening is located 0 to 2 cm from the distal end of the elongate shaft.

17. The embolic protection filter in accordance with claim 10, wherein the transverse opening has a length and a width.

18. The embolic protection filter in accordance with claim 17, wherein the length of the transverse opening is between 0.5 and 3.0 cm.

19. An embolic protection system, comprising:
the first occlusive device including an elongate shaft having a proximal end and a distal end, a lumen extending at least in part through the elongate shaft, an occlusive member disposed proximate the distal end of the elongate shaft, and the shaft having a generally transversely disposed opening;
a second occlusive device including an elongate shaft having a proximal end and a distal end, a lumen extending at least in part through the elongate shaft, and an occlusive member disposed proximate the distal end; and
a second occlusive member being disposed proximate the distal end of the elongate shaft of the second occlusive device, and being disposed at least in part within the lumen of the first occlusive member.

20. The embolic protection system in accordance with claim 19, wherein the first occlusive member includes a filter.

21. The embolic protection system in accordance with claim 19, wherein the second occlusive member includes a balloon in fluid communication with the lumen of the second occlusive device.

22. The embolic protection system in accordance with claim 19, the second occlusive member being disposed within the lumen of the first occlusive device.

23. The embolic protection system in accordance with claim 19, wherein the elongate member of the second occlusive device includes a hypotube.

24. The embolic protection system of claim 23, wherein the outer diameter of the elongate member of the second occlusive device is between 0.010 to 0.015 inches.

25. The embolic protection system of claim 24, wherein the outer diameter of the elongate member of the second occlusive device is approximately 0.012 inches.

26. The embolic protection filter in accordance with claim 19, wherein the shaft comprises the hypotube having an outer diameter of 0.015 to 0.020 inches.

27. The embolic protection filter in accordance with claim 26, wherein the outer diameter of the shaft is 0.016 to 0.019 inches.

28. The embolic protection filter in accordance with claim 27, wherein the outer diameter of the shaft is 0.018 inches.

29. The embolic protection filter in accordance with claim 19, wherein the transverse opening is located 0 to 5 cm from the distal end of the shaft.

30. The embolic protection filter in accordance with claim 29, wherein the transverse opening is located 0 to 3 cm from the distal end of the elongate shaft.

31. The embolic protection filter in accordance with claim 30, wherein the transverse opening is located 0 to 2 cm from the distal end of the elongate shaft.

32. The embolic protection filter in accordance with claim 19, wherein the transverse opening has a length and a width.

33. The embolic protection filter in accordance with claim 32, wherein the length of the transverse opening is between 0.5 and 3.0 cm.
